(19) United States
(12) Patent Application Publication
(10) Pub. No.: US 2005/0119603 A1
(43) Pub. Date: Jun. 2, 2005

(54) Surgical implant for accessing cerebrospinal fluid and method of surgically inserting same

(76) Inventors: Steven Michael Kuhlman, Richland, MI (US); Matthew Crosby Flegal, Kalamazoo, MI (US); Donna Jean Waggoner, Kalamazoo, MI (US); Allen Edwin Buhl, Portage, MI (US); Karl Edward Kappenman, Kalamazoo, MI (US); Clayton Lynn Hadick, Richland, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/980,630

(22) Filed: Nov. 3, 2004

Related U.S. Application Data
(60) Provisional application No. 60/517,315, filed on Nov. 4, 2003.

(51) Int. Cl. A61M 5/00

(52) U.S. Cl. 604/8

(57) ABSTRACT

A surgical implant and method of surgically inserting the implant into the skull of a patient to gain access to cerebrospinal fluid within the lateral ventricles. The implant includes an annular housing defining thereon a flared base and a tubular member which projects outwardly from the housing. The implant is embedded in the skull such that the tubular member projects into a predefined opening in the skull and the base is positioned adjacent the outer surface of the skull. The implant is secured to the skull with adhesive which is applied to the base member. The implant defines an opening which extends therethrough, and serves a guide channel for directing a surgical instrument into the lateral ventricle.

### FIELD OF THE INVENTION

This invention generally relates to an implant for gaining access to cerebrospinal fluid (csf) in the brain, and in particular relates to a surgical implant which serves as a guide for directing a surgical instrument, such as a needle, into the lateral ventricle of the brain.

### BACKGROUND OF THE INVENTION

It is often necessary for research and treatment purposes to have access to cerebrospinal fluid from conscious patients or subjects. For example, by sampling or collecting cerebrospinal fluid, the progression of various brain diseases, infections, or other ailments can be monitored on a regular basis. In research, cerebrospinal fluid sampling is often required to monitor drug levels as well as to monitor changes in physiological parameters in the cerebrospinal fluid. Further, it is often desirable or necessary to administer therapeutic agents directly into the cerebrospinal fluid to bypass the blood-brain barrier.

Various devices and methods have been developed for the purpose of accessing cerebrospinal fluid in humans and animals. One such device is a guide cannula which is intended for implanting within the skull of a canine. One or more of these guide cannulas are secured in the skull of the animal and extend to touch the surface of the dura mater on the surface of the brain so that each of the guides is aligned (but not in contact) with one of the lateral ventricles of the brain. These guides are implanted for the purpose of permitting repeated sampling of cerebrospinal fluid over a predetermined span of time, and thus the guides are left within the skull of the animal and are accessed via a collection needle placed through the skin and muscle located above the respective guides following a surgical-style preparation of the skin over the guides. The needle is inserted into the guide cannula and is guided thereby into the corresponding lateral ventricle to collect cerebrospinal fluid. One of the disadvantages of this arrangement is that the guide cannula locks to the skull of the animal with screw-threads, which can cause difficulty with respect to successfully aligning the needle guide in relation to the lateral ventricle. Further, the screw-threads often result in improper placement of the guide cannula when the sloped surface of the skull catches the threads and pulls the implant out of proper alignment.

The present invention is directed to an implant or needle guide for accessing cerebrospinal fluid from the brain which overcomes or at least minimizes the disadvantages of known devices. The implant includes an upper head or housing having a flange-like base which projects sidewardly from the housing and is fixed to the skull, and a tube or stalk which projects through a hole formed in the skull. In a preferred embodiment, the head and tube together define a lumen which serves as a guide for a surgical instrument, such as a needle. The implant is positioned in the skull over one of the lateral ventricles in the brain based upon predetermined coordinates with the implant head located subcutaneously on the skull, so that when a needle is inserted into the lumen, the implant precisely guides the needle into the lateral ventricle for collection of cerebrospinal fluid or for dosing therapeutic agents directly into the cerebrospinal fluid.

The implant is dimensioned so that when properly positioned in the skull, the free end of the tube is spaced from, and does not penetrate, the lateral ventricle. Thus, the implant itself makes no direct contact with the ventricle. This lack of contact with the ventricle advantageously maintains sterility with repeated use. Further, the flange-like base positioned on the outer surface of the skull allows accurate and reliable positioning of the implant via adhesive such as surgical glue and/or surgical resin, which causes less skull trauma. The free end of the stalk or tube is blunt and rests upon or is disposed closely adjacent the dura mater located beneath the skull which also results in less trauma to the patient, as compared with the above-discussed guide which includes a pointed lower edge which penetrates the dura mater.

Other objects and purposes of the invention will be apparent to persons familiar with devices of this type upon reading the following description and inspecting the accompanying drawings.

### BRIEF DESCRIPTION OF THE DRAWINGS

FIG. 1 is a front elevational view of the implant according to the invention.

FIG. 2 is a plan view of the implant.

FIG. 3 is a cross-sectional view of the implant taken generally along line 3-3 in FIG. 2.

FIG. 4 is a vertical cross-sectional view of the brain of an animal, such as a canine.

FIG. 5 is an enlarged, fragmentary, vertical cross-sectional view of the brain of FIG. 4, with a pair of implants in position in the skull.

FIG. 6 is a vertical cross-sectional view of the brain of a human.

FIG. 7 is an enlarged, fragmentary, vertical, cross-sectional view of the brain of FIG. 6, with a pair of implants in position in the skull.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the arrangement and designated parts thereof. Said terminology will include the words specifically mentioned, derivatives thereof, and words of similar import.

### DETAILED DESCRIPTION

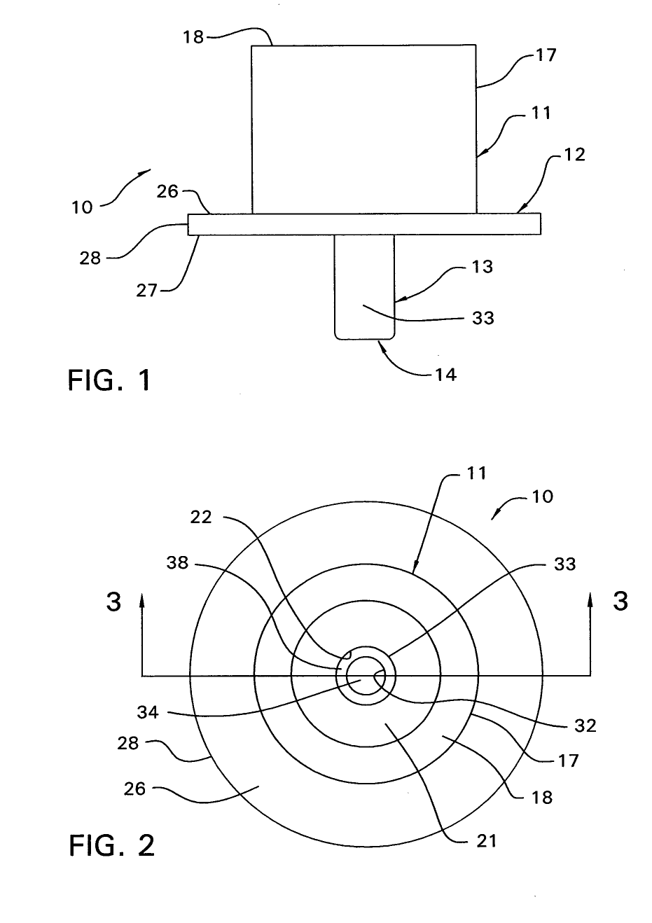

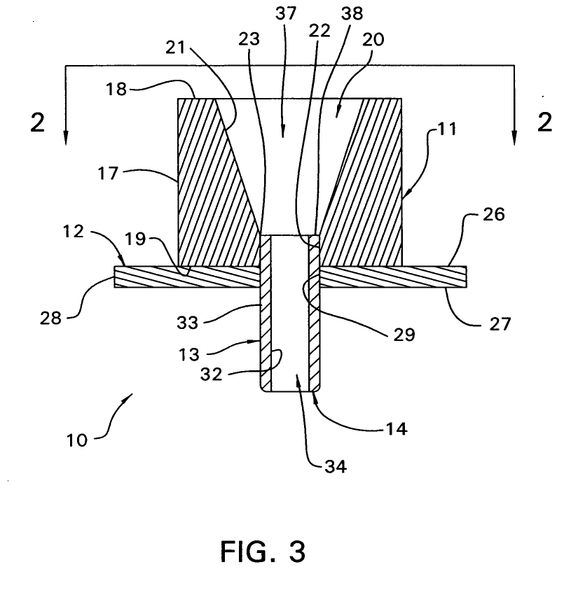

Referring to FIGS. 1-3, an implant or guide 10 is illustrated according to the present invention. The implant 10 generally includes a rigid head or housing 11, a flange-like base 12 positioned at one end of the housing 11, and a tube or stalk 13 which depends downwardly from the base 12 and terminates in a lower free end 14.

Upper housing 11 is generally annular in shape, and defines an outer cylindrical and generally vertically oriented surface 17, a generally planar and annular upper surface 18 which in the illustrated embodiment is generally perpendicular to surface 17, and a generally planar and annular lower surface 19 which is spaced downwardly from and is generally parallel to upper surface 18. Housing 11 has an opening 20 which projects completely therethrough, and is defined by an inner annular surface 21 which is tapered or funnel-shaped when viewed in cross-section as in FIG. 3, and an inner cylindrical surface 22 which adjoins surface 21 at a transition point 23 and projects downwardly therefrom. Surface 22 is generally parallel to outer surface 17.

Base 12 is a thin and flexible plate-like member, and defines thereon generally planar, parallel and annular upper and lower surfaces 26 and 27, and a generally vertically oriented side surface 28 which interconnects surfaces 26 and 27. Base 12 defines therein an opening 29 which projects completely through the thickness of base 12 as defined between the surfaces 26 and 27.

Tube 13 is generally cylindrical in shape and defines inner and outer cylindrical and generally parallel surfaces 32 and 33. Inner surface 32 defines an opening 34 which extends completely through tube 13. As shown in FIG. 3, the outer diameter of tube 13 as well as the diameter of opening 29 of base 12 are similar in dimension to the diameter of the lower portion of housing opening 20 as defined by lower surface 22. Dimensioning the implant components in this manner allows same to be assembled to one another without the use of adhesives or other fasteners. More specifically, the implant 10 is assembled by inserting the tube 13 downwardly through the upper portion of the housing opening 20, and then forcing the tube 13 into the lower constricted part of the opening 20 until an uppermost end 38 of tube 13 is positioned generally at the transition point 23. The base 12 is then fitted over the lower free end 14 of tube 13 until same engages and abuts the lower surface 19 of housing 11. The tube 13 thus effectively serves as a fastening device which secures housing 11 and base 12 to one another through a force-fit arrangement. However, adhesive may be used to further secure housing 11, base 12 and tube 13 to one another, if desirable or necessary. Further, it will be appreciated that housing 11, base 12 and tube 13 may alternatively be secured to one another through a mechanical interlock arrangement.

When housing 11, base 12 and tube 13 are assembled to one another as discussed above, opening 20 of housing 11 and opening 34 of tube 13 together define a lumen 37 which extends through the implant 10 and serves as a guide channel through which a surgical instrument can be inserted.

It will be appreciated that housing 11 and base 12 can instead be constructed as a unitary component, for example by milling a suitably sized cylinder to define base 12. Housing 11, base 12 and tube 13 may also be constructed as a unitary, one-piece component.

In the illustrated embodiment, housing 11, base 12 and tube 13 are constructed of surgical-grade stainless steel. However, these components may alternatively be constructed of non-reactive, injection-molded rigid plastic, resin or titanium.

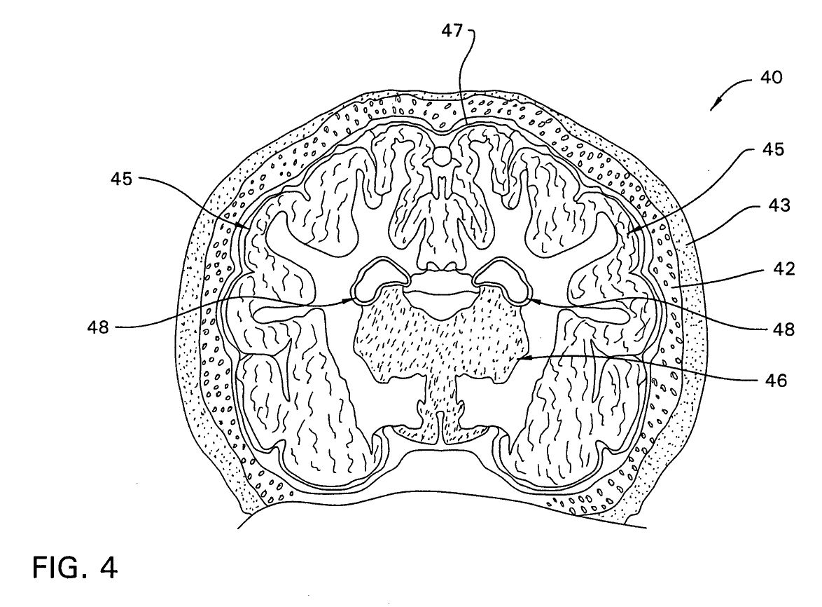

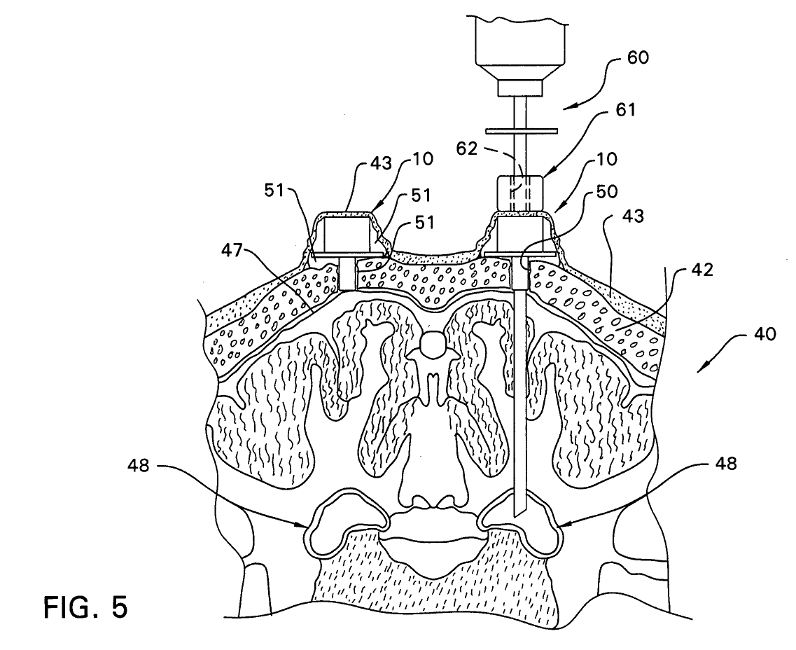

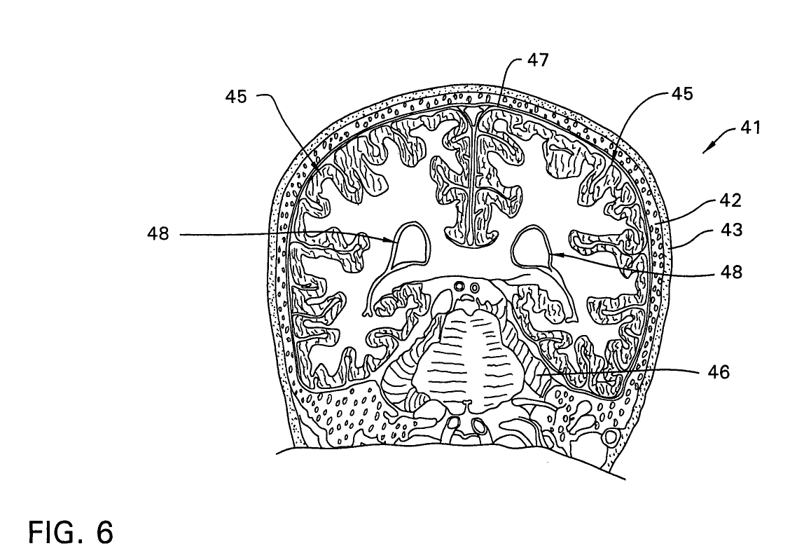

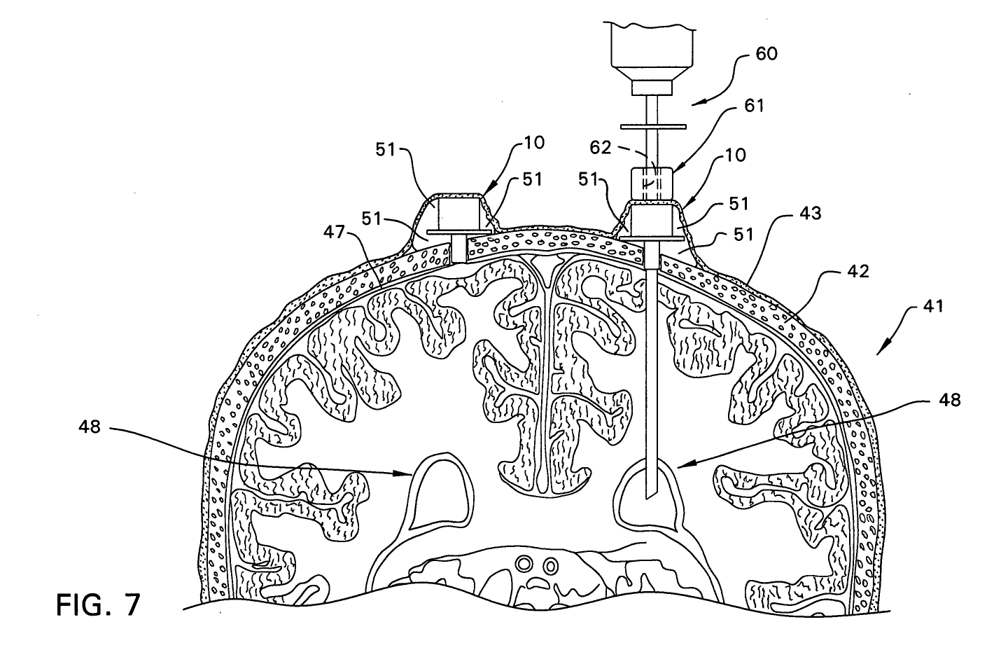

FIGS. 4 and 5 illustrate a brain 40 of an animal, such as a canine and FIGS. 6 and 7 illustrate the brain 41 of a human. The same reference numbers are used in these drawings to refer to the same or similar parts of brains 40 and 41. The brain 40, 41 is contained within the skull 42 covered by skin 43, and includes three primary parts, the cerebrum which is defined by the cerebral hemispheres 45, the cerebellum 46 and the spinal cord or brain stem (not shown). A thick and fibrous membrane called the dura mater 47 lines the interior of the skull 42. The most common sites for accessing cerebrospinal fluid in the brain are the lateral ventricles 48, which are respectively located in the lower and inner parts of the cerebral hemispheres 45 and contain cerebrospinal fluid therein.

The guide 10 according to the invention is implanted into the brain 40, 41 of a patient as follows, with reference to FIGS. 5 and 7. The anesthetized patient is placed in a conventional stereotaxic apparatus and aseptically prepped for surgery. A longitudinal midline incision is made over the center of the skull 42, and dissection is employed to extend the incision down to the surface of the skull 42. The musculature is then laterally peeled away from the skull 42 to clear the area for the implant 10. Using predetermined brain coordinates, a bone drill positioned in the stereotaxic apparatus is used to drill a hole 50 in the skull 42 of the same or similar diameter as the tube 13 at the appropriate position above one of the lateral ventricles 48. The drill is removed, and a syringe and needle, such as manufactured by the Hamilton Company, is assembled to the implant 10 by inserting the needle through the lumen 37 of the implant 10. This assembly is then mounted to the stereotaxic apparatus and the needle advanced into the hole 50 in the skull 42 until cerebrospinal fluid is obtained. The needle is then backed off until same has exited the lateral ventricle 48, and the implant 10 is advanced down the needle until the tube 13 is located about halfway into the hole 50 in the skull 42. Surgical adhesive or gel 51, such as cyanoacrylate gel, is then applied to the exposed upper portion of the tube 13 and to lower surface 27 and the side edge 28 of base 12, and the implant 10 is then advanced down the needle and into position in the skull 42. In this regard, the implant 10 is properly positioned when the lower free end 14 of tube 13 penetrates the skull 42 and rests on the surface of the dura mater 47. The lower free end 14 of the tube 13 is preferably rounded or blunt, so as not to damage or otherwise cause trauma to the skull 42 and the membranes beneath the skull 42.

The surgical gel 51 serves to anchor the implant 10 in place and provides a build-up of material below the housing 11 and base 12 to fill the gap created by the natural curvature of the skull 42. This build-up of surgical gel 51 is necessary since the orientation of the base 12 should preferably be parallel with the horizontal to ensure proper guidance of the surgical instrument into the respective lateral ventricle 48. The enlarged base 12 of the implant 10 advantageously provides an edge or lip which is gripped by the adhesive 51 to firmly lock the implant 10 in the proper angular orientation relative to the skull 42. Additional implants 10 can then be placed within the skull 42 at other coordinates, as shown in FIGS. 5 and 7. Once the implant 10 is in position, an infusion of radio-opaque dye can be infused via the implant 10 and observed with fluoroscopy to confirm the proper directional orientation of tube 13 relative to the respective ventricle 48.

The incision is then closed, and after the skin 43 is fully closed over the skull 42 but prior to the patient's recovery from the anesthesia, a collection needle is inserted into the implant 10 and slowly advanced until cerebrospinal fluid begins welling up in the needle. This needle depth, as measured between the skin 43 to the point at which sufficient fluid flow is achieved, is recorded for the particular implant 10 and is subsequently used to provide the proper needle depth for post-surgical collections or dosings. These recorded needle depths constitute default depths for the particular implants 10 and needles are either specially cut to length for accessing fluid or dosing, or suitable spacers are utilized on standard needles to provide the proper penetration depth as discussed below.

When sampling of cerebrospinal fluid is desirable or necessary, the skull 42 of the patient is felt with the fingers in order to locate the bump or nodule created by the upper housing 11 of the implant 10 under the skin 43. Using standard aseptic practices, a collection needle 60 (FIGS. 5 and 7) is then pushed through the skin 43 and into the lumen 37 of the implant 10 and is guided downwardly by the tapered surface 21 of housing 11 and into the opening 34 of the tube 13. As the needle 60 is advanced, the tube 13 then guides the needle 60 as same penetrates the brain and ultimately enters the lateral ventricle 48. Cerebrospinal fluid is then withdrawn from the lateral ventricle 48 with the needle 60. As discussed above, an appropriately-sized spacer 61 defining a through-hole 62 therein may be utilized in conjunction with needle 60 to ensure proper insertion depth into the ventricle 48. The same procedure is utilized when dosing of a drug or drugs is desirable or necessary, except that a dosing needle is utilized instead of a collection needle and serves to deliver a drug directly into the cerebrospinal fluid located within the lateral ventricle 48.

It will be appreciated that the implant 10 according to the invention may be utilized with humans and canines as discussed above, and the dimensions thereof will be based upon the brain size and structure of the particular animal. In this regard, the implant 10 is also usable with animals other than canines, such as rabbits and non-human primates. When utilized in a human, typical dimensions of the implant 10 are as follows: the upper housing has an outer diameter of approximately 8-10 mm, and a height of approximately 4-6 mm; the base 12 has an outer diameter of approximately 10-12 mm; and the tube 13 projects outwardly from the base 12 over a distance of about 6-8 mm. When used in a canine, typical dimensions of the implant 10 are as follows: the upper housing 11 has an outer diameter of approximately 5 mm, and a height of approximately 4 mm; the base 12 has an outer diameter of approximately 8 mm; and the tube 13 projects outwardly from the base 12 over a distance of about 2-4 mm. When determining the dimensions of the implant 10 for a particular species, the tube 13 should preferably have a length sufficient to permit the tube 13 to penetrate the skull so that the lower free end 14 thereof rests on or adjacent the dura mater 47.

It will be appreciated that the tapered surface 21 defined in the housing 11 enables easy insertion of a surgical instrument into the implant 10. However, the surface 21 need not necessarily be tapered as shown, and the opening may instead be cylindrical, for example.

It will also be appreciated that the implant 10 may be utilized to guide a surgical instrument, such as a needle, into the left ventricle as discussed above, but may alternatively be used to insert a surgical instrument, such as a piezoelectric crystal typically used for obtaining a pressure reading. Further, the implant 10 may be used to insert a fiber-optic camera into the ventricle to visualize same.

Further, the implant 10 according to the invention may also be utilized as a temporary port for accessing cerebrospinal fluid from a patient or subject. In this regard, the implant would require a septum or other penetrable barrier at the upper open end of housing 11 so as to create a closed access port, and the skin would then not be closed over the implant. The implant as modified in this manner could be used for research purposes or for treating injuries.

Although a particular preferred embodiment of the invention has been disclosed for illustrative purposes, it will be recognized that variations or modifications of the disclosed apparatus, including the rearrangement of parts, lie within the scope of the present invention.

1. An implant for accessing cerebrospinal fluid from a lateral ventricle located within a brain enclosed by a skull, said implant comprising a housing part, an elongate tubular member, and a flange fixed to one end of said housing part between said housing part and said tubular member, said flange having an outer dimension which is larger than an outer dimension of said housing part such that said flange projects sidewardly beyond said housing part for mounting on the outer surface of the skull with said housing part projecting outwardly from the outer surface of the skull and said tubular member projecting at least partially into the skull, and a lumen extending through said implant and at one end opening through said housing part and at an opposite end opening through a free end of said tubular member, said lumen defining a guide channel for directing a surgical instrument inserted into said implant into the lateral ventricle for accessing cerebrospinal fluid therein.

2. The implant of claim 1 wherein said implant is free of screw-threads.

3. The implant of claim 1 wherein said free end of said tubular member has a blunt shape to avoid trauma to the skull and/or membranes located beneath the skull.

4. The implant of claim 1 wherein said housing part is annular and defines therein a funnel-shaped opening which extends through said housing part and has its largest dimension located at an end of said housing part opposite said one end of said housing part, said tubular member defines an elongate opening therein which extends through said tubular member, said opening of said housing part and said opening of said tubular member being coaxially aligned with one another and together defining said lumen.

5. The implant of claim 4 wherein an end of said tubular member opposite said free end thereof is disposed within said opening of said housing part adjacent said flange such that said housing part and said flange are disposed in surrounding relation with said tubular member.

6. An implant for positioning subcutaneously on the skull of a patient generally above a lateral ventricle of the brain, said implant comprising a rigid head located subcutaneously and having a generally flat base member at one end thereof which is configured for positioning atop the skull, both said head and base member being free of screw-threads and said base member defining an edge portion which projects sidewardly from an outer surface of said head to define a gripping area for adhesive used to secure the base member to the outer surface of the skull, and a rigid tubular member projecting outwardly from a side of said base member opposite said head and into the skull for guiding a surgical instrument into the lateral ventricle.

7. The implant of claim 6 wherein said head and said tubular member together define an opening which extends through said implant for receiving and guiding a surgical instrument into the lateral ventricle.

8. The implant of claim 6 wherein said tubular member has a free end spaced from said base member, said free end having a rounded shape.

9. The implant of claim 6 wherein said head and said tubular member together define a guide channel which extends through said implant and opens at opposite ends thereof through said head and a free end of said tubular member, respectively, and a portion of said guide channel defined by said head is generally funnel-shaped to assist in insertion of a surgical instrument into said implant.

10. The implant of claim 6 wherein said head is annular in shape and said base member projects sidewardly from a lower end thereof, said base member having a diameter which is greater than a diameter of said head to define an annular lip disposed in surrounding relation with said head for gripping adhesive.

11. A method of surgically inserting an implant into the skull of a patient, said method comprising the steps of:
providing an implant having a housing part, a base member fixed to one end of said housing part and projecting outwardly therefrom to define a lip at said one end, and a tubular member projecting outwardly from a side of said base member opposite said housing part, said implant defining therein a lumen which extends through said implant and which at one end opens through said housing part and at an opposite ends opens through a free end of said tubular member;
locating an area of the skull generally above the lateral ventricle using predetermined coordinates;
forming an opening in the skull at said area;
inserting the tubular member into said opening and positioning said base member adjacent the outer surface of the skull; and
securing said implant to the skull by applying adhesive to said base member such that the adhesive grippingly engages said lip and the skull.

12. The method of claim 11 wherein said step of securing includes securing said implant to the skull without the use of screw-threads.

13. The method of claim 11 wherein said step of securing includes securing said implant to the skull solely with adhesive.

14. The method of claim 13 wherein said step of securing includes positioning said base member immediately adjacent the outer surface of the skull and orienting said base member such that same is horizontal.

15. The method of claim 14 wherein said step of securing includes building up adhesive under at least a portion of said base member to orient said base member horizontally.

16. A method of surgically inserting an implant into the skull of a patient for accessing cerebrospinal fluid from a lateral ventricle of the brain, said method comprising:
providing an implant having a head, a base flange projecting sidewardly from one end of said head, and an elongate tube projecting outwardly from said base flange with said base flange being disposed between said head and said tube, said implant defining a lumen therein which projects through said implant and serves as a guide into the lateral ventricle;
locating the proper area of the skull to access a lateral ventricle based upon predetermined coordinates;
forming an opening in the skull at said area;
embedding the implant in the skull such that said tube projects into said opening and said base flange is disposed adjacent the outer surface of the skull; and
fixing said implant to the skull by applying adhesive to said base flange, without the use of screw-threads.

17. The method of claim 16 wherein said step of embedding includes inserting said tube into said skull opening until a free end of the tube is disposed adjacent, but does not penetrate, the dura mater located inside the skull.

18. The method of claim 16 wherein said step of forming includes forming an opening in the skull which is similar in diameter to the outer diameter of said tube.

19. The method of claim 16 wherein said step of fixing includes orienting said base flange so as to be disposed in a horizontal orientation, and building up adhesive under said base flange to fix same in said horizontal orientation relative to the outer surface of the skull.

20. The method of claim 16 wherein said base flange defines a lip for gripping engagement with said adhesive.
